(19) United States
(12) Patent Application Publication
(10) Pub. No.: US 2011/0224215 A1
(43) Pub. Date: Sep. 15, 2011

(54) PRE-SURGICAL TREATMENT

(75) Inventor: Jack A. DeJovin, New Brunswick, NJ (US)

(73) Assignee: GALDERMA LABORATORIES, INC., Fort Worth, TX (US)

(21) Appl. No.: 12/809,354

(22) PCT Filed: Dec. 17, 2008
(86) PCT No.: PCT/US08/13797

§ 371 Date: May 31, 2011

Related U.S. Application Data
(60) Provisional application No. 61/015,912, filed on Dec. 21, 2007.

(51) Int. Cl. A61K 31/498; A61P 41/00; A61P 7/04

(52) U.S. Cl. 514/249

(57) ABSTRACT

The invention relates to a method of reducing post-surgical bleeding and/or bruising in a patient scheduled to be subjected to a surgical procedure and a patient that was subjected to a surgical procedure. The method involves topically applying to the area and the surrounding area of skin of the patient where the surgical procedure will be or was performed, a pharmaceutical composition comprising an effective amount of brimonidine or a pharmaceutically acceptable salt thereof, prior to performing the surgical procedure and/or following the surgical procedure.

### CROSS-REFERENCE TO RELATED APPLICATION

This application asserts priority from U.S. Provisional Patent Application Ser. No. 61/015,912, the contents of which are incorporated herein by reference.

### BACKGROUND OF THE INVENTION

When a patient undergoes a surgical procedure, the recovery period is often more painful and complicated than the surgical procedure itself. Several factors affect the healing process, such as the age and physical condition of the patient, or the amount of bleeding and/or bruising during and after the surgical procedure. While factors such as the age and physical condition of the patient cannot be changed, it would be beneficial to find a way to reduce bleeding and/or bruising around the site of the surgical procedure.

It has been reported that patients receiving prophylactic dosages of brimonidine prior to undergoing LASIK surgery for primary myopia/myopic astigmatism or hyperopic astigmatism have less occurrence of subconjunctival hemorrhage and less hyperemia. See Norden, “Effect of prophylactic brimonidine on bleeding complications and flap adherence after laser in situ keratomileusis,” J Refract Surg. 2002 July-August; 18(4):468-71. Subconjunctival hemorrhage occurs when a blood vessel in the conjuctiva is ruptured or broken. A ruptured or broken blood vessel causes blood to leak into the space between the conjuctiva and the sclera. The blood can visibly be seen in the white portion of the eye.

Brimonidine is a known α2 adrenergic receptor agonist. The effects of α2 adrenergic receptor agonists, however, are highly unpredictable in the art. For example, an α2 adrenergic receptor agonist may cause vasodilation of blood vessels in certain tissue, vasoconstriction in another tissue, or have no effect at all.

Brimonidine tartrate in aqueous solution (0.15% and 0.20%) has been known for ophthalmic use for many years. It is sold by Allergan under the name ALPHAGAN® P.

However, use of brimonidine on the skin is relatively new. It has recently been discovered that brimonidine tartrate is also useful in treating erythema caused by rosacea. Creams and gels containing brimonidine tartrate have been disclosed in the following U.S. patent applications: U.S. Ser. No. 10/853,585 to DeJovin, et al. (now U.S. Pat. No. 7,439,241); U.S. Ser. No. 10/626,037 to Scherer; and U.S. Ser. No. 10/763,807 to Shanler, et al.

### SUMMARY OF THE INVENTION

The invention relates to a method for reducing post-surgical bleeding and/or bruising in a patient scheduled to be subjected to a surgical procedure, such as a surgical incision or a laser procedure. The method includes topically applying to the area of the skin of the patient where a surgical procedure that causes bleeding and/or bruising is scheduled to be performed, a pharmaceutical composition comprising an effective amount of brimonidine or a pharmaceutically acceptable salt thereof, prior to performing the surgical procedure.

In one embodiment, the pharmaceutical composition is applied at least 10 minutes, and no more than 120 minutes prior to the surgical procedure. In anther embodiment, the pharmaceutical composition is applied at least 15 minutes, and no more than 60 minutes prior to the surgical procedure.

The pharmaceutical composition comprises a pharmaceutical carrier selected from the group consisting of a cream, a gel, an emulsion, and ointment, a solution, and a pre-medicated bandage. The pharmaceutical composition is preferably a gel or a cream. In gel or cream compositions, the brimonidine or pharmaceutically acceptable salt thereof is present in an amount from about 0.1% by weight to about 10% by weight.

Another aspect of the invention relates to a method for reducing bleeding and/or bruising in a patient that was subjected to a surgical procedure, such as a surgical incisions or a laser procedure. The method includes topically applying to the area of the skin of the patient where the surgical procedure was performed, a pharmaceutical composition comprising an effective amount of brimonidine or a pharmaceutically acceptable salt thereof.

The pharmaceutical composition comprises a pharmaceutical carrier selected from the group consisting of a cream, a gel, an emulsion, and ointment, a solution, and a pre-medicated bandage. The pharmaceutical composition is preferably a gel or a cream. In gel or cream compositions, the brimonidine or pharmaceutically acceptable salt thereof is present in an amount from about 0.1% by weight to about 1% by weight in the cream or gel.

In a preferred embodiment, the pharmaceutical composition is applied immediately following the surgical procedure.

### DETAILED DESCRIPTION OF THE INVENTION

The pharmaceutical composition is applied topically to the skin a sufficient time prior to the surgery to reduce bleeding and/or bruising. In a preferred embodiment, the pharmaceutical composition is applied at least 10 minutes prior to the surgical procedure, more preferably at least 15 minutes prior to the surgical procedure, and most preferably at least 30 minutes prior to the surgical procedure. The pharmaceutical composition is preferably applied no more than about 60 minutes prior to the surgical procedure, and most preferably no more than about 120 minutes prior to the surgical procedure.

The pharmaceutical composition is applied topically to the area of the skin where the incision or procedure is scheduled to be performed, and the surrounding area of the skin. The incision or procedure may be performed on any area of the body such as the head, chest, arms, legs, abdomen, etc. For example, a procedure may be performed on the face of a patient.

The area of the skin where the pharmaceutical composition is applied is any area of the skin that would contribute to reducing bleeding and/or bruising following a surgical procedure. For example, the pharmaceutical composition may be applied over the site where the incision will be made and a three inch radius around the site where the incision will be made.

The pharmaceutical composition of the invention is topically applied in any conventional manner known in the art. For example, the compositions are applied by cotton swab or applicator stick, or by simply spreading a formulation of the invention onto the affected area with gloved fingers.

The composition may also be applied using a bandage pre-medicated with the pharmaceutical composition or a bandage soaked in a solution of the pharmaceutical composition. The bandage may be left on the skin for a sufficient amount of time so that bleeding and/or bruising are reduced following a surgical procedure. For example, the bandage may be left on the skin for a minimum of about 10 minutes, more preferably about 15 minutes. The bandage may also be left on the skin for a maximum of about 90 minutes, and more preferably about 30 minutes.

The amount of a topical formulation of the invention applied to the affected skin area is any amount sufficient to reduce bleeding and/or bruising following a surgical procedure. For example, the minimum amount of topical formulation applied may be about 0.0001 g/cm² of skin surface area, more preferably about 0.001 g/cm² of skin surface area. The maximum amount of topical formulation applied may be about 0.01 g/cm² of skin surface area, more preferably about 0.007 g/cm² of skin surface area, and most preferably about 0.003 g/cm² of skin surface area.

Surgical procedures are any procedures that cause bleeding and/or bruising. Surgical procedures include, for example, surgical incisions and laser procedures. Surgical incisions involve puncturing the skin with an instrument such as a scalpel, knife, razor, syringe, laser or the like. Laser procedures are conducted using electromagnetic radiation of a frequency that can cut, or vaporize skin tissue and blood vessels.

The method can be used prior to any surgical procedure that causes bleeding and/or bruising. The method can be used prior to plastic surgery and procedures, for example, laser resurfacing, mole or blemish removal, Botox injections, breast implantation, abdominoplasty, face-lift, rhinoplasty, liposuction, and the like. The method can also be used prior to non-cosmetic surgeries and procedures such as gastric bypass, appendectomy, C-section, gallbladder removal, angioplasty, open heart surgery, kidney transplantation, brain surgery, and the like.

In another application, a pharmaceutical composition of the invention can be applied to the skin of a person prior to shaving. The pharmaceutical composition may be applied any time prior to shaving to be effective for preventing bleeding and/or bruising caused by the razor. For example, the pharmaceutical composition may be applied at least 15 minutes prior to shaving.

The pharmaceutical composition may be applied to any area of the skin that is shaved, such as the face and legs. Application of the pharmaceutical composition prior to shaving will significantly reduce, if not eliminate, bleeding and bruising of cuts or nicks caused by the razor.

### Pharmaceutical Composition

The pharmaceutical composition comprises brimonidine, i.e., 5-bromo-6-(2-imidazolidinylideneamino)quinoxaline, or a pharmaceutically acceptable salt thereof.

Pharmaceutically acceptable salts include those salts of brimonidine that are safe for topical use in patients, and that possess the desired biological activity. Pharmaceutically acceptable acid addition salts include salts of basic groups present in brimonidine. Pharmaceutically acceptable acid addition salts include, but are not limited to, chloride, bromide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. For a review on pharmaceutically acceptable salts see BERGE ET AL., 66 J. PHARM. SCI. 1-19 (1977), incorporated herein by reference.

A preferred pharmaceutically acceptable salt of brimonidine is brimonidine tartrate. Its structure is shown below.

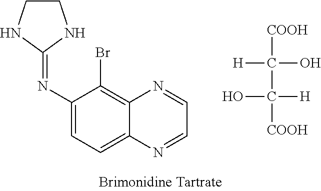

The pharmaceutical compositions of the invention contain brimonidine or a pharmaceutically acceptable salt thereof in an amount therapeutically effective to reduce bleeding and/or bruising when applied prior to surgery. The actual effective amounts of brimonidine or a pharmaceutically acceptable salt thereof will vary according to, for example, the particulate sites of administration, the method of application, the length of time the composition is in contact with the skin, and the subject being treated (e.g. age, gender, skin type, etc.). Such effective amounts can be readily determined by physicians and clinicians during pre-clinical and clinical trials, and by surgeons prior to surgery.

For example, pharmaceutical compositions of the invention may contain a minimum amount of brimonidine or a pharmaceutically acceptable salt thereof of about 0.01% by weight, more preferably about 0.10% by weight, and most preferably about 0.17% by weight. The pharmaceutical compositions may contain a maximum amount of brimonidine or a pharmaceutically acceptable salt thereof of about 10% by weight, more preferably about 5% by weight, more preferably about 1% by weight, and most preferably about 0.19% by weight.

The pharmaceutical compositions also include a pharmaceutically acceptable topical carrier. A pharmaceutically acceptable topical carrier is any pharmaceutically acceptable formulation that can be applied to the skin surface for topical, dermal, intradermal, or transdermal delivery of a pharmaceutical or medicament. The combination of a pharmaceutically acceptable topical carrier and a compound of the invention is termed a topical formulation of the invention. Topical formulations of the invention are prepared by mixing a compound of the invention with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 19th ed. 1995); Ghosh, T. K.; et al. TRANSDERMAL AND TOPICAL DRUG DELIVERY SYSTEMS (1997), both of which are hereby incorporated herein by reference.

### Pharmaceutically Acceptable Topical Carriers

The topical carriers useful for topical delivery of compounds of the invention can be any carrier known in the art for topically administering pharmaceuticals, for example, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions, such as standard ophthalmic preparations.

### Emulsions, Gels, and Ointments as Topical Carriers

In a preferred embodiment, the topical carrier used to deliver a compound of the invention is an emulsion, gel, or ointment. Emulsions, such as creams and lotions are suitable topical formulations for use in the invention. An emulsion is a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In another embodiment, the pharmaceutically acceptable carrier is a gel. Gels are semisolid systems that contain suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention may be two-phase or single-phase systems. Some examples of suitable gels are disclosed in REMINGTON: THE SCIENCE AND PRACTICE OF PHARMACY 1517-1518 (Alfonso R. Gennaro ed. 19th ed. 1995), wherein the description of gels is incorporated herein by reference. Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002), wherein the description of gels is incorporated herein by reference.

Gelling agents, that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.).

“CARBOPOL®” is one of numerous cross-linked acrylic acid polymers that are given the general adopted name carbomer. “Carbomer” is the USP designation for various polymeric acids that are dispersible but insoluble in water. When the acid dispersion is neutralized with a base a clear, stable gel is formed. The preferred carbomer is Carbomer 934P because it is physiologically inert and is not a primary irritant or sensitizer. Other carbomers include 910, 940, 941, and 1342.

Carbomers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling agents include hydroxyethylcellulose, cellulose gum, MVE/MA decadiene crosspolymer, PVM/MA copolymer, or a combination thereof.

In a preferred embodiment, the minimum amount of gelling agent in the composition is about 0.5%, more preferably, about 0.75%, and most preferably about 1%.

In another preferred embodiment, the maximum amount of gelling agent in the composition is about 2%, more preferably about 1.75%, and most preferably about 1.5%.

In another preferred embodiment, the topical carrier used to deliver a compound of the invention is an ointment. Ointments are oleaginous semisolids that contain little if any water. Preferably, the ointment is hydrocarbon based, such as a wax, petrolatum, or gelled mineral oil. Suitable ointments for use in the invention are well known in the art and are disclosed in REMINGTON: THE SCIENCE AND PRACTICE OF PHARMACY 1585-1591 (Alfonso R. Gennaro ed. 19th ed. 1995), hereby incorporated herein by reference.

The pH of the pharmaceutical carrier is adjusted with a base such as sodium hydroxide or potassium hydroxide. The minimum pH of the carrier is about 5, preferably 5.5, and most preferably 6.2 when the carrier is diluted by a factor of ten. The maximum pH of the carrier is about 7.5, preferably 7, and most preferably 6.8 when the carrier is diluted by a factor of ten.

The pH values given above are those that occur if the composition is diluted with water by a factor of ten. It is not necessary to dilute the composition by a factor of ten in order to obtain a pH value. In practice, the composition may be diluted by any value that permits pH to be measured. For example, the composition may be diluted by a factor of about five to about twenty. The pH will vary slightly depending upon the dilution factor.

### Aqueous Topical Formulations of the Invention

In another embodiment, the topical carrier used in the topical formulations of the invention is an aqueous solution or suspension, preferably, an aqueous solution. Well-known ophthalmic solutions and suspensions are suitable topical carriers for use in the invention. Suitable aqueous topical formulations for use in the invention are disclosed in REMINGTON: THE SCIENCE AND PRACTICE OF PHARMACY 1563-1576 (Alfonso R. Gennaro ed. 19th ed. 1995), hereby incorporated herein by reference. Other suitable aqueous topical carrier systems are disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); U.S. Pat. No. 6,465,464 (issued Oct. 15, 2002), wherein the description of aqueous topical carrier systems is incorporated herein by reference.

The pH of the aqueous topical formulations of the invention are preferably within the range of from about 6 to about 8, more preferably, of from about 6.3 to about 6.5. To stabilize the pH, preferably, an effective amount of a buffer is included. In one embodiment, the buffering agent is present in the aqueous topical formulation in an amount of from about 0.05 to about 1 weight percent of the formulation. Acids or bases can be used to adjust the pH as needed. Suitable buffering agents are listed below.

Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation.

Preferably, the aqueous topical formulations of the invention have a viscosity in the range of from about 15 cps to about 25 cps. The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

In a preferred embodiment, the aqueous topical formulation of the invention is isotonic saline comprising a preservative, such as benzalkonium chloride or chlorine dioxide, a viscosity-adjusting agent, such as polyvinyl alcohol, and a buffer system such as sodium citrate and citric acid.

### Excipients

The compositions of the invention may include pharmaceutically acceptable excipients including, but not limited to, protective agents, adsorbents, demulcents, emollients, preservatives, anti-oxidants, moisturizers, buffering agents, solubilizing agents, skin-penetration agents, and surfactants. Pharmaceutically acceptable excipients are listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), wherein the description of excipients is incorporated herein by reference.

Suitable protective agents and/or cosmetic agents, and adsorbents include, but are not limited to, dusting powders, zinc sterate, collodion, dimethicone, silicones, zinc carbonate, aloe vera gel and other aloe products, vitamin E oil, allatoin, petrolatum, titanium dioxide, and zinc oxide. The preferred protective agent is titanium dioxide.

In a preferred embodiment, the minimum amount of cosmetic agent in the composition is about 0.01%, more preferably, about 0.025%, and most preferably about 0.05%.

In another preferred embodiment, the maximum amount of cosmetic agent in the composition is about 0.15%, more preferably about 0.1%, and most preferably about 0.075%.

Suitable demulcents include, but are not limited to, benzoin, hydroxypropyl cellulose, hydroxypropyl methylcellulose, and polyvinyl alcohol.

Suitable emollients include, but are not limited to, animal and vegetable fats and oils, myristyl alcohol, alum, and aluminum acetate.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; parabens such as methylparaben, ethylparaben, propylparaben, and butylparaben; antibacterial esters, for example, esters of parahydroxybenzoic acid; and other anti-microbial agents such as chlorhexidine, chlorocresol, benzoic acid, polymyxin, and phenoxyethanol. The preferred preservatives are methylparaben and phenoxyethanol.

In a preferred embodiment, the minimum amount of preservative in the composition is about 0.1%, more preferably, about 0.2%, and most preferably about 0.3%.

In another preferred embodiment, the maximum amount of preservative in the composition is about 1%, more preferably about 0.75%, and most preferably about 0.5%.

Suitable antioxidants include, but are not limited to, ascorbic acid and its esters, sodium bisulfite, butylated hydroxytoluene, butylated hydroxyanisole, tocopherols, and chelating agents like EDTA and citric acid.

Suitable moisturizers include, but are not limited to, glycerin, sorbitol, polyethylene glycols, urea, and propylene glycol. The preferred moisturizer is glycerin.

In a preferred embodiment, the minimum amount of moisturizer in the composition is about 2%, more preferably, about 3.5%, and most preferably about 4.5%.

In another preferred embodiment, the maximum amount of moisturizer in the composition is about 10%, more preferably about 8%, and most preferably about 6%.

Suitable buffering agents for use with the invention include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methylpyrrolidone. The preferred skin-penetrating agent is propylene glycol.

In a preferred embodiment, the minimum amount of skin-penetrating agent in the composition is about 2%, more preferably, about 3.5%, and most preferably about 4.5%.

In another preferred embodiment, the maximum amount of skin-penetrating agent in the composition is about 10%, more preferably about 8%, and most preferably about 6%.

### Pharmaceutical Additives

The topical formulations of the invention may comprise brimonidine as the only active ingredient or may comprise other active ingredients. Examples of other active ingredients include, but are not limited to, topical corticosteroids and other anti-inflammatory agents, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

Another aspect of the invention relates to a method for reducing bleeding and/or bruising in a patient that was subjected to a surgical procedure. The method involves topically applying to the area of the skin of the patient where a surgical procedure was performed, a pharmaceutical composition comprising an effective amount of brimonidine or a pharmaceutically acceptable salt thereof.

The pharmaceutical composition and methods of application, including, for example, dosage, carrier, etc., are as described above. The pharmaceutical composition may be applied topically to the site of the surgical procedure as soon as possible following the procedure. In a preferred embodiment, the pharmaceutical composition is applied immediately following the surgical procedure.

The methods of the invention may be used prior to and/or following a surgical procedure as necessary to reduce bleeding and/or bruising caused by a surgical procedure.

### EXAMPLES

Examples have been set forth below for the purposes of illustration and to describe the best mode of the invention at the present time. The scope of the invention is not to be in any way limited by the examples set forth herein.

### Example 1

### Synthesis of Brimonidine (5-Bromoquinoxalin-6-yl)-(4,5-dihydro-1H-imidazol-2-yl)-amine

To a stirred solution of 6-amino-5-bromoquinoxaline hydrobromide (10 g) in distilled water (150 ml) is added thiophosgene (3 ml). The solution is stirred for two hours at room temperature and the resultant precipitate is collected by filtration, washed with water, and dried to afford 5-bromo-6-isothiocyanato-quinoxaline.

The 5-bromo-6-isothiocyanato-quinoxaline (3.5 g) is directly dissolved in benzene (400 ml) and added dropwise to a well-stirred solution of ethylene diamine (15 g.) in benzene (50 ml). During a period of about two hours, an oil separates as a lower layer. The upper benzene layer is poured off and the oil is washed with diethyl ether and then dissolved in methanol (500 ml). The methanolic solution is refluxed until hydrogen sulfide evolution ceases. The methanolic solution is concentrated in vacuo to a volume of approximately 100 ml upon which a yellow solid precipitates. The precipitate is collected by filtration and recrystallized from methanol to afford of (5-Bromo-quinoxalin-6-yl)-(4,5-dihydro-1H-imidazol-2-yl)-amine: m.p. 250-251° C.

### Example 2

### Synthesis of Brimonidine Tartrate 5-bromo-6-(2-imidazolidinylideneamino) quinoxaline L-tartrate

The tartrate salt of brimonidine can be synthesized by adding (L)-(+)-tartaric acid to a solution of brimonidine in aqueous methanol. The brimonidine tartrate will separate out of solution.

### Example 3

### Gel Formulation

| Ingredient | Weight Percent |
|---|---|
| Brimonidine tartrate | 0.18% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 4

### Cream Formulation

| Ingredient | Weight Percent |
|---|---|
| Brimonidine tartrate | 0.18% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Steary Alcohol | 12.0% |
| Ceteareth-25 | 2.0% |
| Tartaric Acid | 0.001% |
| DI Water | 56.209% |
| TOTAL | 100% |

### Example 5

### Application of Brimonidine Tartrate Cream Before Botox Injection

A patient is scheduled to receive a Botox injection in the forehead between the eyebrows to reduce frown lines. Ten minutes prior to the Botox injection, 0.18% brimonidine tartrate in a cream formulation is applied to the facial skin between the eyebrows with a cotton applicator.

### Example 6

### Application of Brimonidine Tartrate Gel to Facial Skin Prior to Shaving

Fifteen minutes prior to shaving, a man applies 0.10% brimonidine tartrate in a gel formulation to the facial skin where he will shave. The brimonidine tartrate formulation will prevent excessive bleeding if he cuts himself as he's shaving.

### Example 7

### Application of Brimonidine Via a Bandage Prior to a Patient Prior Undergoing an Appendectomy

Thirty minutes prior to undergoing surgery for an appendectomy, a bandage that is soaked in a solution of 0.25% brimonidine is applied to the area of skin where the incision will be made. The bandage is removed immediately before the incision is made. The application of the brimonidine will significantly reduce bleeding and bruising following the surgical procedure.

### Example 8

### Application of Brimonidine Tartrate Cream to a Patient Recovering from an Appendectomy

In another example, a patient is recovering after receiving an appendectomy. 0.40% brimonidine tartrate cream is applied to the area where the surgical procedure was performed.

1. A method for reducing post-surgical bleeding and/or bruising in a patient scheduled to be subjected to a surgical procedure, the method comprising topically applying to the area of the skin of the patient where a surgical procedure that causes bleeding and/or bruising is scheduled to be performed, a pharmaceutical composition comprising an effective amount of brimonidine or a pharmaceutically acceptable salt thereof, prior to performing the surgical procedure.

2. A method according to claim 1, wherein the surgical procedure is a surgical incision or laser procedure.

3. A method according to claim 1, wherein the pharmaceutical composition is applied at least 10 minutes, and no more than 120 minutes prior to the surgical procedure.

4. A method according to claim 3, wherein the pharmaceutical composition is applied at least 15 minutes, and no more than 60 minutes prior to the surgical procedure.

5. A method according to claim 1, wherein the pharmaceutical composition comprises a pharmaceutical carrier selected from the group consisting of a cream, a gel, an emulsion, and ointment, a solution, and a pre-medicated bandage.

6. A method according to claim 5, wherein the pharmaceutical composition is a gel or a cream.

7. A method according to claim 6, wherein the brimonidine or pharmaceutically acceptable salt thereof is present in an amount from about 0.1% by weight to about 10% by weight in the cream or gel.

8. A method for reducing bleeding and/or bruising in a patient that was subjected to a surgical procedure, the method comprising topically applying to the area of the skin of the patient where the surgical procedure was performed, a pharmaceutical composition comprising an effective amount of brimonidine or a pharmaceutically acceptable salt thereof.

9. A method according to claim 8, wherein the surgical procedure is a surgical incision or laser procedure.

10. A method according to claim 8, wherein the pharmaceutical composition comprises a pharmaceutical carrier selected from the group consisting of a cream, a gel, an emulsion, and ointment, a solution, and a pre-medicated bandage.

11. A method according to claim 10, wherein the pharmaceutical composition is a gel or a cream.

12. A method according to claim 11, wherein the brimonidine or pharmaceutically acceptable salt thereof is present in an amount from about 0.1% by weight to about 1% by weight in the cream or gel.

13. A method according to claim 8, wherein the pharmaceutical composition is applied immediately following the surgical procedure.
